(19) United States
(12) Patent Application Publication
(10) Pub. No.: US 2022/0110584 A1
(43) Pub. Date: Apr. 14, 2022

(54) DOSAGE REGIMEN SUPPORTING APPARATUS AND DOSAGE REGIMEN SUPPORTING SYSTEM

(71) Applicant: CANON MEDICAL SYSTEMS CORPORATION, Otawara-shi (JP)

(72) Inventor: Junichi TAKAZAWA, Nasushiobara (JP)

(73) Assignee: CANON MEDICAL SYSTEMS CORPORATION, Otawara-shi (JP)

(21) Appl. No.: 17/495,048

(22) Filed: Oct. 6, 2021

(30) Foreign Application Priority Data
Oct. 9, 2020 (JP) 2020-170971

(51) Int. Cl. A61B 5/00; G16H 20/10

(52) U.S. Cl. CPC A61B 5/4848; G16H 20/10; A61B 5/4839

(57) ABSTRACT

A dosage regimen supporting apparatus according to an embodiment includes processing circuitry. The processing circuitry estimates the symptom of the side effect of a patient as a dosage target of a therapeutic agent and the timing of onset of the symptom in the next dosage period of the therapeutic agent based on biological information or report information of the patient related to the symptom of the side effect and the timing of onset of the symptom, and attribute information of the patient. The processing circuitry outputs a suggestion related to content and a timing of treatment for dealing with the side effect based on the biological information or the report information, and an estimation result.

### CROSS-REFERENCE TO RELATED APPLICATIONS

This application is based upon and claims the benefit of priority from Japanese Patent Application No. 2020-170971, filed on Oct. 9, 2020; the entire contents of which are incorporated herein by reference.

### FIELD

Embodiments described herein relate generally to a dosage regimen supporting apparatus and a dosage regimen supporting system.

### BACKGROUND

Conventionally, in a case of making a treatment plan with an agent such as an anticancer agent, a doctor makes a dosage schedule while considering side effects that occur in a patient. Typically, side effects are different among individual patients, so that it is not easy in some cases to make an appropriate treatment plan while considering side effects that individually occur in a patient.

### BRIEF DESCRIPTION OF THE DRAWINGS

FIG. 1 is a block diagram illustrating an example of a configuration of a dosage regimen supporting system according to an embodiment;

FIG. 2 is a diagram illustrating an example of a dosage regimen support screen according to the embodiment;

FIG. 3 is a diagram illustrating another example of the dosage regimen support screen according to the embodiment;

FIG. 4 is a diagram illustrating an example of the dosage regimen support screen displaying a side effect based on report information according to the embodiment;

FIG. 5 is a diagram for explaining reflection of a treatment plan in a regimen plan sheet according to the embodiment;

FIG. 6 is a diagram illustrating an example of an input screen for the report information according to the embodiment; and

FIG. 7A is a flowchart illustrating an example of a procedure of dosage regimen support processing according to the embodiment.

FIG. 7B is a continuation of the flowchart illustrated in FIG. 7A.

### DETAILED DESCRIPTION

The following describes an embodiment of a dosage regimen supporting apparatus and a dosage regimen supporting system in detail with reference to the drawings.

The dosage regimen supporting apparatus according to the embodiment includes processing circuitry. The processing circuitry acquires attribute information of a patient as a dosage target of a therapeutic agent, and at least one of quantitative biological information of the patient indicating a symptom of a side effect caused by the therapeutic agent and a timing of onset of the symptom, and subjective report information of the patient related to the symptom of the side effect and the timing of onset of the symptom. The processing circuitry estimates the symptom of the side effect of the patient and the timing of onset of the symptom in the next dosage period of the therapeutic agent based on the biological information or the report information, and the attribute information of the patient. The processing circuitry outputs a suggestion about content and a timing of treatment for dealing with the side effect based on the biological information or the report information, and an estimation result.

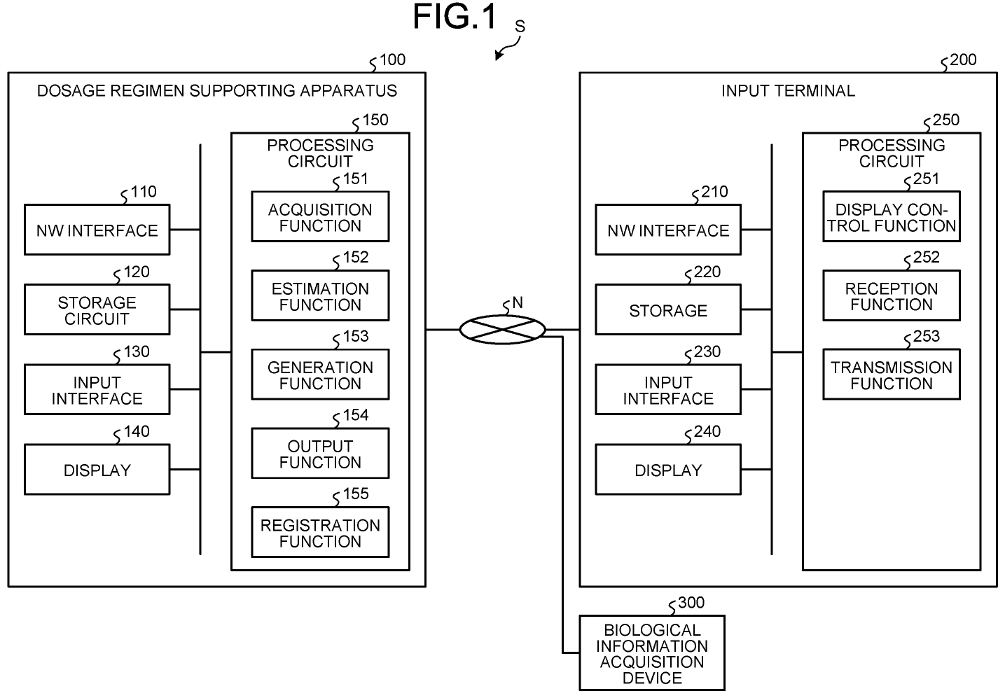

FIG. 1 is a block diagram illustrating an example of a configuration of a dosage regimen supporting system S according to the present embodiment. As illustrated in FIG. 1, the dosage regimen supporting system S includes a dosage regimen supporting apparatus 100, an input terminal 200, and a biological information acquisition device 300. The dosage regimen supporting apparatus 100, the input terminal 200, and the biological information acquisition device 300 are connected to each other via a network N. The network N is the Internet, an in-hospital local area network (LAN), or the like. The biological information acquisition device 300 is not necessarily connected to the dosage regimen supporting apparatus 100, and may be connected to only the input terminal 200, for example. The dosage regimen supporting system S may be connected to various information processing devices such as an electronic medical chart system (not illustrated) via the network N.

The dosage regimen supporting apparatus 100 is assumed to be a computer such as a personal computer (PC), for example. The dosage regimen supporting apparatus 100 is installed in a medical institution such as a hospital, for example.

The dosage regimen supporting apparatus 100 is a device for supporting a doctor and the like to make a dosage regimen of a therapeutic agent for the patient. The therapeutic agent is an agent that is periodically administered to the patient for each period of a predetermined length for multiple times. In the present embodiment, a user of the dosage regimen supporting apparatus 100 is a doctor, for example.

The present embodiment exemplifies a case in which the therapeutic agent is an anticancer agent. For example, in the present embodiment, one dosage period of the anticancer agent includes ten days, and a plurality of dosage periods are assumed to be planned as one set. The one dosage period may also be called “one course”. The length of the dosage period is merely an example, and an application target of the configuration according to the present embodiment is not limited thereto.

Therapeutic agents may be continuously administered during the dosage periods, or the therapeutic agent may be administered once for each dosage period.

The anticancer agent according to the present embodiment is not limited to a chemotherapeutic agent, but includes a molecularly targeted therapeutic agent. The therapeutic agent may be other agents.

The input terminal 200 is used by the patient as a dosage target of the therapeutic agent. The user of the input terminal 200 is the patient. Hereinafter, in the present embodiment, a simple word of “patient” indicates the patient as the dosage target of the therapeutic agent.

The input terminal 200 is, for example, a tablet terminal, a smartphone, a PC, or the like.

The biological information acquisition device 300 is mounted on a body of the patient, and measures biological information of the patient. The biological information acquisition device 300 is, for example, a thermometer, a sphygmomanometer, a pulse oximeter, or the like. The one biological information acquisition device 300 may have a plurality of functions, or a plurality of the biological information acquisition devices 300 may be mounted on the patient. The biological information acquisition device 300 is not necessarily mounted on the body of the patient at all times.

The following describes the dosage regimen supporting apparatus 100, the input terminal 200, and the biological information acquisition device 300.

The dosage regimen supporting apparatus 100 includes a network (NW) interface 110, a storage 120, an input interface 130, a display 140, and a processing circuit 150.

The NW interface 110 is connected to the processing circuit 150, and controls transmission and communication of various kinds of data among the dosage regimen supporting apparatus 100, the input terminal 200, and the biological information acquisition device 300. The NW interface 110 is implemented by a network card, a network adapter, a network interface controller (NIC), and the like.

The storage 120 is connected to the processing circuit 150, and stores various pieces of information to be used in the processing circuit 150. Additionally, in the present embodiment, the storage 120 stores, for example, the attribute information of the patient acquired from the electronic medical chart system and the like, the biological information acquired from the biological information acquisition device 300, the report information about the side effect acquired from the input terminal 200, and the like. The storage 120 also stores a regimen plan sheet used for treatment of the patient. Details about the patient attribute information, the biological information, the report information, and the regimen plan sheet will be described later.

The storage 120 is, for example, implemented by a semiconductor memory element such as a random access memory (RAM) and a flash memory, a hard disk, an optical disc, and the like. The storage 120 is also called a storage unit.

The input interface 130 is implemented by a trackball, a switch button, a mouse, a keyboard, a touch pad for performing input operation by touching an operation surface thereof, a touch screen in which a display screen is integrated with a touch pad, a noncontact input circuit with an optical sensor, a voice input circuit, and the like. The input interface 130 is connected to the processing circuit 150, and converts an input operation received from the user into an electric signal to be output to the processing circuit 150.

In the present application, the input interface 130 is not limited to an input interface including a physical operation component such as a mouse and a keyboard. For example, examples of the input interface 130 include a processing circuit for electric signals that receives an electric signal corresponding to the input operation from an external input appliance disposed separately from the device, and outputs the electric signal to the processing circuit 150.

The display 140 is a liquid crystal display, an organic electro-luminescence (OEL) display, and the like. The input interface 130 and the display 140 may be integrated with each other. For example, the input interface 130 and the display 140 may be implemented by a touch panel. The display 140 is an example of a display unit.

The processing circuit 150 is processing circuitry that implements a function corresponding to each computer program by reading out, from the storage 120, and executing the computer program. The processing circuit 150 according to the present embodiment has an acquisition function 151, an estimation function 152, a generation function 153, an output function 154, and a registration function 155. The acquisition function 151 is an example of an acquisition unit. The estimation function 152 is an example of an estimation unit. The generation function 153 is an example of a generation unit. The output function 154 is an example of an output unit. The registration function 155 is an example of a registration unit.

For example, each of processing functions of the acquisition function 151, the estimation function 152, the generation function 153, the output function 154, and the registration function 155, which are constituent elements of the processing circuit 150, is stored in the storage 120 in a form of a computer-executable program. The processing circuit 150 is processing circuitry. For example, the processing circuit 150 implements a function corresponding to each computer program by reading out, from the storage 120, and executing the computer program. In other words, the processing circuit 150 that has read out each computer program is assumed to have each function illustrated in the processing circuit 150 in FIG. 1. In the description about FIG. 1, a single piece of the processing circuitry is assumed to implement the processing functions executed by the acquisition function 151, the estimation function 152, the generation function 153, the output function 154, and the registration function 155. Alternatively, the processing circuit 150 may include a plurality of independent pieces of the processing circuitry, and the independent piece of the processing circuitry may execute the respective computer programs to implement the respective functions. In the description about FIG. 1, the single storage 120 is assumed to store the computer program corresponding to each processing function. Alternatively, a plurality of storages may be disposed in a distributed manner, and the processing circuit 150 may read out a corresponding computer program from an individual storage.

In the above description, exemplified is a case in which the “processing circuitry” reads out, from the storage, and executes the computer program corresponding to each function, but the embodiment is not limited thereto. In the present embodiment, the phrase of “processing circuitry” means, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). For example, in a case in which the processing circuitry is a CPU, the processing circuitry implements a function by reading out a computer program stored in the storage and executing the computer program. On the other hand, in a case in which the processing circuitry is an ASIC, the function is directly embedded, as a logic circuit, in a circuit of the processing circuitry instead of storing the computer program in the storage 120. Each piece of the processing circuitry according to the present embodiment is not necessarily configured as a single circuit for each piece of the processing circuitry. A plurality of independent circuits may be combined with each other and configured as a piece of the processing circuitry to implement the function. A plurality of constituent elements in FIG. 1 may be further integrated into the one piece of the processing circuitry to implement the function.

The acquisition function 151 acquires the attribute information of the patient as a dosage target of the therapeutic agent, and at least one of the biological information of the patient and the report information of the patient. In the present embodiment, the acquisition function 151 acquires both of the biological information and the report information.

More specifically, the acquisition function 151 acquires the biological information of the patient as a dosage target of the therapeutic agent from the biological information acquisition device 300, the electronic medical chart system, or the like via the NW interface 110.

The biological information is quantitative information indicating the symptom of the side effect caused by the therapeutic agent and the timing of onset of the symptom.

The timing of onset of the symptom means, for example, on what day of one dosage period the symptom has appeared. By way of example, in a case in which the symptom of the side effect is a fever, and a body temperature of the patient exceeds a threshold on the second day of the dosage period of ten days, the second day is the timing of onset of the symptom in the dosage period.

For example, the quantitative information is assumed to be numerical information that is objectively obtained by examining the body of the patient. Specifically, the biological information includes an examination result of at least one of a body temperature, a blood pressure, oxygen saturation, a red blood cell count, a white blood cell count, and a platelet count, and an examination date. The biological information may also include additional information, for example, various kinds of vital data, a result of a blood test, or the like.

The acquisition function 151 also acquires the report information input by the patient from the input terminal 200 via the NW interface 110.

The report information is subjective information of the patient related to the symptom of the side effect of the therapeutic agent and the timing of onset of the symptom. In the present embodiment, the report information is self-reported by being input by the patient through the input terminal 200. By way of example, the report information is information obtained by associating content of the symptom of the side effect, an intensity of the symptom, and a date with each other.

By way of example, the report information includes mental or sensuous information of the patient. In the present embodiment, the report information includes at least one of side effects including “having mental painfulness”, “having no appetite”, “having sleepiness”, and “having swelling of the body”. The side effects to be self-reported as the report information are not limited thereto. The report information may also include a chief complaint of the patient.

The acquisition function 151 also acquires, via the input interface 130, the attribute information of the patient input by the user. Alternatively, the acquisition function 151 may acquire the attribute information of the patient from the electronic medical chart system (not illustrated) via the NW interface 110.

The attribute information of the patient includes, for example, at least one of an age, distinction of sex, a physical constitution, a body mass index (BMI), a type of cancer, a stage of cancer, vital data at the time when treatment is started with an anticancer agent, and a chronic disease of the patient. The attribute information of the patient may also include additional information.

Each of the biological information, the report information, and the attribute information is acquired while being associated with identification information with which the patient can be identified. The identification information is, for example, a patient ID, a patient name, or the like.

The acquisition function 151 associates the acquired biological information, report information, and attribute information with the identification information of the patient to be stored in the storage 120.

In the present embodiment, the acquisition function 151 is assumed to acquire both of the biological information and the report information, but the acquisition function 151 may acquire only one of the biological information and the report information. That is, the acquisition function 151 is assumed to acquire the attribute information of the patient, and at least one of the biological information and the report information.

The acquisition function 151 also acquires, via the input interface 130, an analysis result related to the side effect of the patient input by a user's operation.

In the present embodiment, the analysis result is a result obtained by analyzing, by a doctor, the actual symptom of the side effect of the patient and the timing of onset of the symptom in a previous dosage period. The analysis result may be input as a natural sentence by the doctor, or may be selected by the doctor from among candidates prepared in advance. A specific example of the analysis result will be described later.

The acquisition function 151 sends out the acquired analysis result to the generation function 153.

The acquisition function 151 also acquires, via the input interface 130, a change of the content and the timing of treatment for dealing with the side effect made by the user. The change is, for example, a change of the suggestion related to the content and the timing of treatment for dealing with the side effect in the next dosage period that is displayed on the display 140 by the output function 154 described later. The acquisition function 151 sends out content of the acquired change to the output function 154 and the registration function 155.

The estimation function 152 estimates the symptom of the side effect of the patient and the timing of onset of the symptom in the next dosage period of the therapeutic agent based on the biological information or the report information, and the attribute information of the patient.

More specifically, the estimation function 152 estimates the symptom of the side effect of the patient and the timing of onset of the symptom in the next dosage period of the therapeutic agent based on information related to developments of the side effect of a similar patient that is specified based on the biological information or the report information of the patient, and the attribute information of the patient.

The symptom of the side effect of the patient and the timing of onset of the symptom in the next dosage period estimated by the estimation function 152 are examples of estimation results according to the present embodiment.

The similar patient is a patient having attribute information similar to that of the patient as a current dosage target among other patients to whom the same therapeutic agent as that for the patient as the dosage target of the therapeutic agent was administered in the past. More specifically, at least one of an age, distinction of sex, a physical constitution, a BMI, a type of cancer, a stage of cancer, vital data at the time when treatment is started with the therapeutic agent, and a chronic disease of the similar patient is the same as that of the patient as the dosage target of the therapeutic agent. The similar patient is not limited to a patient to whom the completely same therapeutic agent as that for the patient as the current dosage target is administered, but may be a patient to whom another therapeutic agent the side effect of which is similar to that of the current therapeutic agent is administered.

The information related to developments of the side effect of the similar patient is biological information or report information of the similar patient. Hereinafter, the biological information or report information of the similar patient in the past is referred to as similar patient information.

As the similar patient information, for example, track records of the biological information or report information of the patient as the dosage target of the therapeutic agent in the past may be stored in the storage 120. By way of example, the storage 120 includes a database in which pieces of information about the patient as a current treatment target and patients treated in the past are accumulated. The database stores the identification information, the biological information, and the report information of the patient as a target of a current treatment plan and the other patients treated in the past. The similar patient information may be information that is acquired using a method different from that for the biological information and the report information of the patient as the current treatment target. For example, the similar patient information may be a past treatment track record acquired from the electronic medical chart system and the like.

For example, the estimation function 152 may extract a plurality of pieces of the similar patient information from the storage 120, and may cause a result obtained by averaging symptoms of side effects of the extracted similar patients and timings when the symptoms appear to be the estimation result of the symptom of the side effect of the patient and the timing of onset of the symptom. Alternatively, the estimation function 152 may cause the similar patient information of one similar patient to be the estimation result of the symptom of the side effect of the patient and the timing of onset of the symptom, the one similar patient having the attribute information most similar to that of the current patient among patients to whom the agent was administered in the past, the biological information or the report information of the patients being registered in the storage 120.

The estimation function 152 uses, for the estimation, the similar patient information in the same dosage period as the next dosage period of the patient as the current dosage target of the therapeutic agent. For example, in a case in which administration of the therapeutic agent to a patient is to be started, the next dosage period of the patient will be the first dosage period. In this case, the estimation function 152 extracts, from the storage 120, the biological information or the report information in the first dosage period of a past patient similar to the patient as the similar patient information to be used for the estimation.

A method of estimating the symptom of the side effect of the patient and the timing of onset of the symptom by the estimation function 152 is not limited to the method described above. For example, the estimation function 152 may estimate the symptom of the side effect of the patient and the timing of onset of the symptom using a pre-learned model obtained by associating the attribute information of the patient to whom the agent was administered in the past with the biological information or the report information of the patient. The pre-learned model is, for example, a pre-learned model generated by deep learning of a neural network and the like. As a method of deep learning, a convolutional neural network (CNN), a recurrent neural network (RNN), and the like can be applied, but the method is not limited thereto.

Characteristics such as a timing of onset of the side effect with the anticancer agent, severity of the symptom, and duration of the symptom are different among individuals, but these characteristics may be similar, with high possibility, between patients having similar pieces of attribute information. Thus, the estimation function 152 estimates the symptom of the side effect of the patient and the timing of onset of the symptom in the next dosage period based on the similar patient information.

In the present embodiment, for the second and subsequent dosage periods, the estimation function 152 basically uses, as the estimation result for the next dosage period, the biological information or the report information in a previous dosage period of the patient instead of the similar patient information. This is because, in a case in which the anticancer agent is administered to the same person over a plurality of dosage periods, the timing of onset of the side effect, severity of the symptom, and duration of the symptom are typically similar among the dosage periods as a characteristic of the side effect of the anticancer agent.

However, this characteristic cannot be applied to all patients. Even about the same person, the timing of onset of the side effect, severity of the symptom, duration of the symptom, and the like may be largely different among different dosage periods.

Thus, in a case in which the actual symptom of the side effect of the patient or the timing of onset of the symptom in the previous dosage period is different from the actual symptom of the side effect of the patient or the timing of onset of the symptom in a dosage period previous to the previous dosage period by a predetermined reference value or more, the estimation function 152 estimates the symptom of the side effect of the patient and the timing of onset of the symptom in the next dosage period based on the similar patient information.

That is, in this case, the estimation function 152 estimates the symptom of the side effect of the patient and the timing of onset of the symptom in the next dosage period based on the information related to developments of the side effect of the similar patient. As a condition for the similar patient, the estimation function 152 may add the condition that a change in the symptom of the side effect or the timing of onset of the symptom among the dosage periods is equal to or larger than the reference value. For example, in a case in which the previous dosage period is the second dosage period, and the dosage period previous to the previous dosage period is the first dosage period, the estimation function 152 may assume, as the similar patient, a patient having a change in the symptom of the side effect or the timing of onset of the symptom equal to or larger than the reference value between the first dosage period and the second dosage period among past patients having the attribute information similar to that of the patient as the dosage target.

In a case in which the actual symptom of the side effect of the patient and the timing of onset of the symptom in the previous dosage period is not different from the actual symptom of the side effect of the patient or the timing of onset of the symptom in the dosage period previous to the previous dosage period by the predetermined reference value or more, the estimation function 152 estimates the symptom of the side effect of the patient and the timing of onset of the symptom in the next dosage period based on the previous biological information or report information of the patient. That is, in this case, the estimation function 152 estimates the symptom of the side effect of the patient and the timing of onset of the symptom in the next dosage period based on the actual symptom of the side effect of the patient and the timing of onset of the symptom in the previous dosage period.

The estimation function 152 may directly use the actual symptom of the side effect of the patient and the timing of onset of the symptom in the previous dosage period as the estimation result of the symptom of the side effect of the patient and the timing of onset of the symptom for the next dosage period, or may generate an estimation result while considering aggravation of the symptom of the side effect due to an increased number of dosage periods. For example, even if a dose of the anticancer agent is not changed, severity of the symptom of the side effect may be increased due to a decline in the physical strength of the patient, accumulation of loads on the body, and the like as the number of times of administration increases. Thus, the estimation function 152 may estimate the severity of the symptom in the next dosage period to be higher than that of the symptom in the previous dosage period. The estimation function 152 may also estimate the duration of the symptom in the next dosage period to be longer than the duration of the symptom in the previous dosage period.

A standard for using which of the similar patient information, and the previous biological information or report information of the patient for estimation is not limited. Alternatively, the user may determine to use, for the estimation, which of the similar patient information, and the previous biological information or report information of the patient.

The estimation function 152 also outputs the estimation result to the generation function 153 and the output function 154.

The generation function 153 generates a suggestion related to content and a timing of treatment for dealing with the side effect in the next dosage period based on the biological information or the report information, and the estimation result obtained by the estimation function 152. Dealing with the side effect means, for example, to prevent the symptom of the side effect from aggravating, or to alleviate aggravation of the symptom of the side effect.

The treatment for dealing with the side effect is not limited to medical treatment. For example, in a case in which a side effect of “having swelling of the body” appears, the patient may wear compression socks for relieving swelling of legs. In a case in which a side effect of “having mental painfulness” appears, visitors may be restricted as a countermeasure. As an example of a medical countermeasure, a medicine is administered. In a case in which the side effect is a fever, for example, an antifebrile may be administered as an example of the countermeasure. The countermeasure includes a change in a dose of the anticancer agent, or a change in a type of the anticancer agent to be administered.

For example, the generation function 153 generates a suggestion about the countermeasure based on an analysis result related to the actual symptom of the side effect of the patient and the timing of onset of the symptom in the previous dosage period, and the biological information or the report information of the patient in the previous dosage period.

In a case in which the analysis result is input by the doctor as a natural sentence, the generation function 153 has a function of converting the natural sentence into structured data. As the function of converting the natural sentence into structured data, for example, a pre-learned model by deep learning may be used, but the embodiment is not limited thereto.

In the present embodiment, the generation function 153 uses the analysis result from the doctor acquired by the acquisition function 151 to generate the suggestion. The biological information or the report information of the patient in the previous dosage period represents the actual symptom of the side effect of the patient and the timing of onset of the symptom in the previous dosage period.

For example, the generation function 153 may output the content and the timing of treatment for dealing with the side effect in the next dosage period by inputting the analysis result, and the biological information or the report information to the pre-learned model obtained by associating the analysis result, the biological information or the report information, and the content and the timing of treatment for dealing with the side effect in the next dosage period with each other. The method of generating the suggestion is not limited thereto. For example, the generation function 153 may use a database and the like obtained by associating the analysis result, the biological information or the report information, and the content and the timing of treatment for dealing with the side effect in the next dosage period with each other.

The generation function 153 sends out the generated suggestion to the output function 154 and the registration function 155.

The output function 154 outputs the suggestion related to the content and the timing of treatment for dealing with the side effect in the next dosage period. The suggestion is a suggestion generated by the generation function 153, and is based on the biological information or the report information, and the estimation result obtained by the estimation function 152. In the present embodiment, “output” encompasses causing a display unit such as the display 140 to display information, or outputting information to another information processing device and the like. The present embodiment describes a case of causing the display 140 to display information as an example of the output, but a method of the output is not limited thereto.

The output function 154 also causes the display 140 to display the estimation result obtained by the estimation function 152 on a time-series basis in a comparable manner with the biological information or the report information of the patient. The biological information or the report information of the patient indicates the actual symptom of the side effect of the patient and the timing of onset of the symptom.

For example, the output function 154 causes the display 140 to display the symptom of the side effect and the timing of onset of the symptom estimated by the estimation function 152, and the actual symptom of the side effect of the patient and the timing of onset of the symptom in the previous dosage period in a comparable manner.

Specifically, the output function 154 causes the display 140 to display a dosage regimen support screen for supporting the doctor to generate a dosage regimen in the next dosage period.

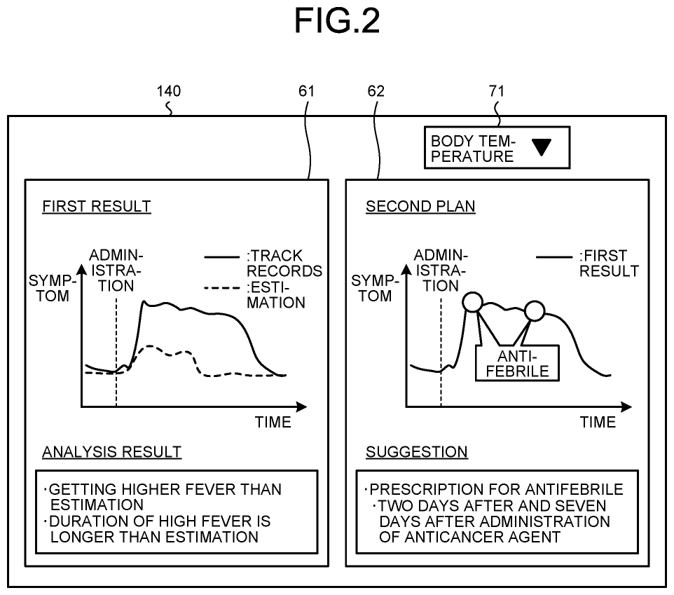

FIG. 2 is a diagram illustrating an example of the dosage regimen support screen according to the present embodiment. As illustrated in FIG. 2, the dosage regimen support screen is displayed on the display 140. By way of example, the dosage regimen support screen includes a first area 61, a second area 62, and a list box 71. FIG. 2 illustrates the dosage regimen support screen at the time when a dosage regimen for the second dosage period is made after the first dosage period ends.

The list box 71 is an operation unit by which the user can select the biological information or the report information as a display target. In the example illustrated in FIG. 2, a “body temperature” as a piece of the biological information is selected by the user.

The list box 71 may also be an operation unit by which a type of the side effect can be selected. In this case, for example, the type of the side effect such as “fever” may be an option. Means for selecting the biological information or the report information as the display target, or the type of the side effect by the user is not limited to the list box 71, but may be a menu screen or a search screen, for example.

In FIG. 2, it is assumed that the anticancer agent is administered to the patient on the second day after each dosage period is started.

The first area 61 is a screen region in which information about the side effect in a past dosage period is displayed. The second area 62 is a screen region in which information about the side effect in the next dosage period is displayed.

More specifically, the output function 154 displays, in the first area 61, the estimated symptom of the side effect and timing of onset of the symptom, and the actual symptom of the side effect of the patient and the timing of onset of the symptom in the previous dosage period in a comparable manner with each other on a time-series basis. That is, estimation and track records of the symptom of the side effect of the patient and the timing of onset of the symptom in the previous dosage period are displayed in the first area 61. In FIG. 2, the previous dosage period is the first dosage period, so that estimation and track records in the first dosage period are displayed on a time-series basis in a comparable manner with each other.

Estimated values displayed in the first area 61 in FIG. 2 are estimated from the similar patient information by the estimation function 152.

In the second area 62, an estimation result of the symptom of the side effect of the patient and the timing of onset of the symptom in the next dosage period are displayed. As described above, for the second and subsequent dosage periods, the estimation function 152 basically uses the biological information or the report information of the patient in the previous dosage period as the estimation result for the next dosage period instead of the similar patient information. Thus, in the second area 62 illustrated in FIG. 2, track records of the symptom of the side effect of the patient in the first dosage period and the timing of onset of the symptom are displayed.

Graphs illustrated in the first area 61 and the second area 62 in FIG. 2 indicate severity of symptom on the vertical axis, and time on the horizontal axis. In FIG. 2, the “body temperature” is selected in the list box 71, so that the graphs illustrated in the first area 61 and the second area 62 represent time-series changes of a value of the body temperature among pieces of the biological information of the patient. In this case, the “symptom” on the vertical axis is the value of the body temperature indicating a degree of fever. The time on the horizontal axis indicates the number of days in the dosage period.

Specifically, the graph illustrated in the first area 61 of FIG. 2 represents track record values of a measurement result of the body temperature of the patient for each day in the first dosage period, and estimated values of a measurement result of the body temperature of the patient for each day estimated before the first dosage period is started.

The graph illustrated in the second area 62 represents track record values of a measurement result of the body temperature of the patient for each day in the first dosage period.

Before the first dosage period is started, track records are yet to be present, so that only estimated values generated from the similar patient information are displayed in the first area 61. In this case, the estimated values generated from the similar patient information are displayed also in the second area 62. Alternatively, before the first dosage period is started, only the second area 62 may be displayed on the dosage regimen support screen.

In the first area 61, an analysis result related to the actual symptom of the side effect of the patient and the timing of onset of the symptom in the previous dosage period is displayed. In the example illustrated in FIG. 2, the analysis result is “getting a higher fever than estimation” and “duration of a high fever is longer than estimation”. The analysis result is a result input by the doctor as the user with reference to the graph of the estimated values and the track record values displayed in the first area 61, for example.

The analysis result may be input by the doctor as a natural sentence, or may be selected by the doctor from candidates prepared in advance. For example, the output function 154 may display a pull-down menu of the candidates for the analysis result, and may dispose a selection column with which the doctor can select the analysis result on the dosage regimen support screen.

In the second area 62, a suggestion related to the content and the timing of treatment for dealing with the side effect in the next dosage period is displayed. For example, the suggestion is a suggestion generated by the generation function 153 based on the analysis result input to an analysis result column of the first area 61. In the example illustrated in FIG. 2, it is suggested that an antifebrile is prescribed two days after and seven days after administration of the anticancer agent. That is, the content of treatment in FIG. 2 is “prescription for the antifebrile”, and the timing of treatment is “two days after and seven days after administration of the anticancer agent”.

The output function 154 also superimposes the timing of treatment in the suggestion on the time-series changes of the symptom of the side effect of the patient in the previous dosage period, or the time-series changes of the symptom of the side effect of the patient based on the estimation result, so as to be displayed on the display 140. For example, as illustrated in FIG. 2, the output function 154 may display the content and the timing of treatment so as to be superimposed on the graph in the second area 62. This display is an example of a treatment plan for the next dosage period according to the present embodiment.

As described above, the generation function 153 may determine a medicine to be prescribed and a timing of prescription by using the pre-learned model, for example. The analysis result of “getting a higher fever than estimation” and “duration of a high fever is longer than estimation” may be previously associated with “prescription for the antifebrile”, and stored in the storage 120. The timing of prescription for the antifebrile is determined based on a timing when the fever reaches a peak on the graph of the estimated values displayed in the second area 62, for example.

In FIG. 2, the timing when the fever reaches the peak is identical to the timing for prescribing the antifebrile, but the timing for prescribing the antifebrile may be a timing obtained by subtracting a time lag until an effect of the antifebrile is exhibited from the time at which a high fever is estimated to reach the peak.

The user can change the suggestion displayed on the dosage regimen support screen. For example, the user may change the content or the timing of treatment such as the “antifebrile” or “two days after, seven days after” in the suggestion displayed in the second area 62 as a sentence. The user may change the suggestion by moving display of treatment on the graph displayed in the second area 62 by drag and drop.

Next, the following describes the dosage regimen support screen in a case of targeting the third and subsequent dosage periods.

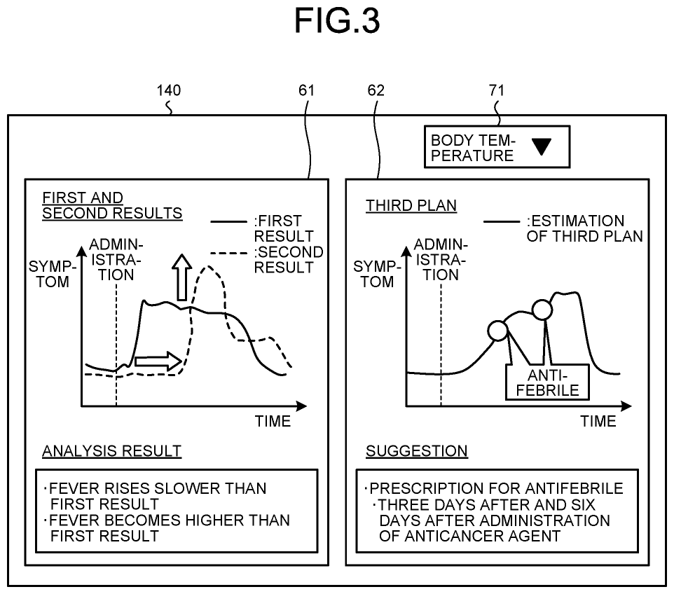

FIG. 3 is a diagram illustrating another example of the dosage regimen support screen according to the present embodiment. FIG. 3 illustrates the dosage regimen support screen at the time when a dosage regimen for the third dosage period is made after the second dosage period ends.

In a case in which two or more dosage periods have already elapsed, as illustrated in FIG. 3, track records of the symptom of the side effect of the patient and the timing of onset of the symptom in the previous dosage period and the dosage period previous to the previous dosage period are displayed in a comparable manner with each other on a time-series basis in the first area 61.

In the example illustrated in FIG. 3, it is assumed that the actual symptom of the side effect of the patient or the timing of onset of the symptom in the second dosage period is different from the actual symptom of the side effect of the patient or the timing of onset of the symptom in the first dosage period by a predetermined reference value or more. In this case, as described above, the estimation function 152 estimates the symptom of the side effect of the patient and the timing of onset of the symptom in the third dosage period based on the information related to developments of the side effect of a similar patient instead of the track records of the patient in the previous dosage period. In this case, as illustrated in FIG. 3, the output function 154 displays, in the second area 62, an estimation result of the symptom of the side effect of the patient and the timing of onset of the symptom in the third dosage period based on the similar patient information.

In a case in which the actual symptom of the side effect of the patient or the timing of onset of the symptom in the second dosage period is not different from the actual symptom of the side effect of the patient or the timing of onset of the symptom in the first dosage period by the predetermined reference value or more, the output function 154 displays, in the second area 62, an estimation result based on actual measured values of the actual symptom of the side effect of the patient and the timing of onset of the symptom in the two dosage periods.

For planning of the third and subsequent dosage periods, the output function 154 causes a suggestion based on the analysis result to be displayed similarly to planning of the second dosage period described above with reference to FIG. 2.

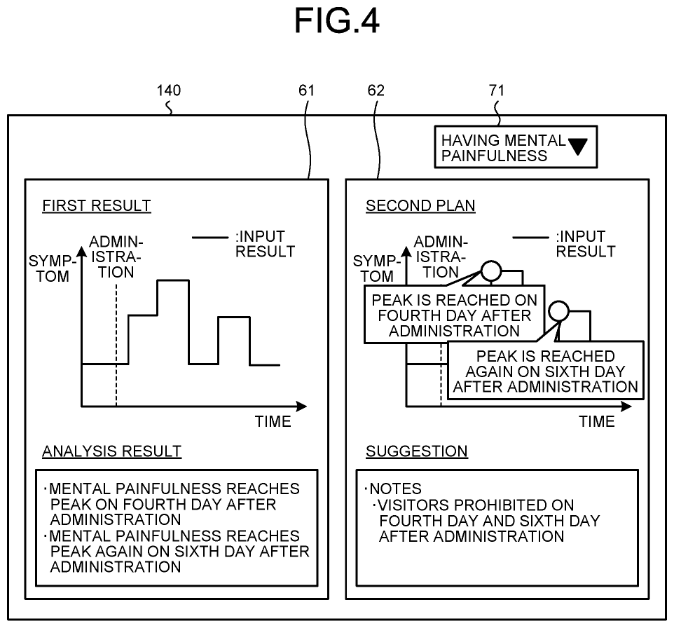

In the description about FIGS. 2 and 3, exemplified is the side effect based on the biological information. The following describes an example of the side effect based on the report information with reference to FIG. 4.

FIG. 4 is a diagram illustrating an example of the dosage regimen support screen displaying the side effect based on the report information according to the present embodiment. FIG. 4 illustrates the dosage regimen support screen at the time when the dosage regimen for the second dosage period is made after the first dosage period ends.

In the example illustrated in FIG. 4, the side effect of “having mental painfulness” is selected in the list box 71 as a display target by the user.

In FIG. 4, track records of the report information of the patient regarding the side effect of “having mental painfulness” in the first dosage period are displayed in the first area 61 on a time-series basis. More specifically, the graph illustrated in the first area of FIG. 4 represents a degree of mental painfulness for each day. As illustrated in the analysis result, in the first dosage period in FIG. 4, the mental painfulness of the patient reaches a peak on the fourth day after administration of the anticancer agent, is lowered once, and reaches the peak again on the sixth day.

In the example illustrated in FIG. 4, only track record values of the report information are displayed in the first area 61, but estimated values may also be displayed. Alternatively, the estimation function 152 does not necessarily generate estimated values for the report information.

In the second area 62, an estimation result of the timing of onset of the side effect based on the report information of the patient in the next dosage period is displayed. In the example illustrated in FIG. 4, track records of the symptom of the side effect and the timing of onset of the symptom in the first dosage period of the patient are displayed in the second area 62 as estimated values in the second dosage period. The output function 154 may display, in the second area 62, the estimation result that the estimation function 152 has estimated while considering aggravation of the symptom of the side effect due to an increased number of dosage periods.

In the example illustrated in FIG. 4, the output function 154 displays a suggestion for restricting visitors on a day when the mental painfulness of the patient is estimated to reach the peak, as treatment in the second dosage period generated by the generation function 153. This suggestion can also be changed by the user.

The output function 154 also causes the display 140 to display a regimen screen, which will be described later. The regimen screen is a screen for viewing or editing the regimen plan sheet.

Returning to FIG. 1, the registration function 155 registers a treatment plan based on the suggestion in the regimen plan sheet related to administration of the anticancer agent. Herein, the treatment plan is the content and the timing of treatment for dealing with the side effect in the next dosage period. In a case in which the user changes the suggestion, the registration function 155 registers the content or the timing of treatment changed by the user in the regimen plan sheet.

The regimen plan sheet is a treatment plan sheet on a time-series basis obtained by combining administration of all agents used for treatment. The regimen plan sheet is also simply called a regimen. Typically, in a case in which the anticancer agent is administered to the patient, the doctor makes a treatment plan using the regimen plan sheet. A standard treatment plan is determined as a guideline depending on a type or a stage of cancer, but the doctor adjusts the treatment plan for individual patients, and makes the regimen plan sheet suitable for each patient. The dosage regimen supporting apparatus 100 according to the present embodiment supports the doctor to make the regimen plan sheet.

More specifically, the registration function 155 updates, based on the suggestion generated by the generation function 153 or a result obtained by changing the suggestion by the user, the regimen plan sheet that is generated based on the guideline.

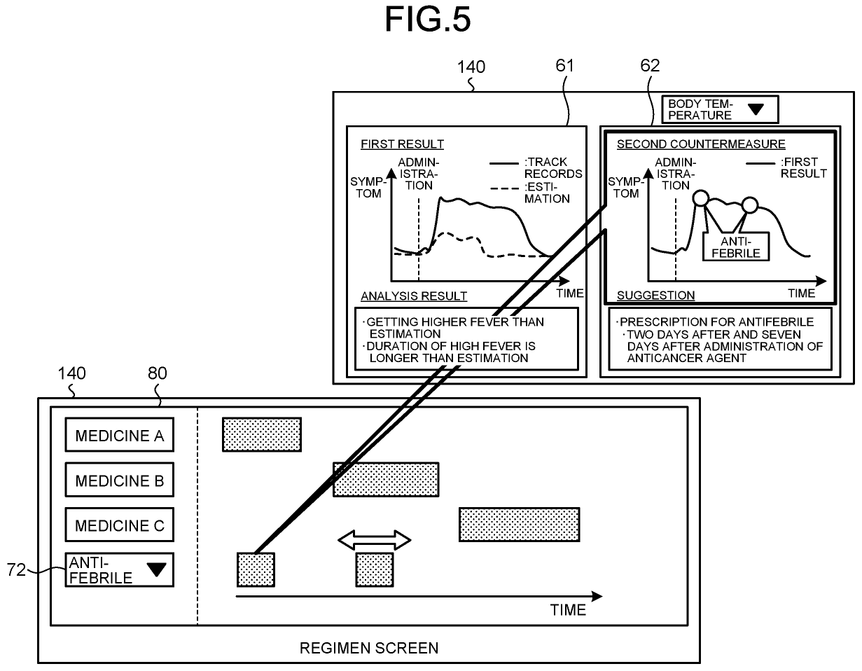

FIG. 5 is a diagram for explaining reflection of the treatment plan in the regimen plan sheet according to the embodiment. In FIG. 5, a regimen screen 80 is displayed on the display 140 by the output function 154.

As illustrated in FIG. 5, the registration function 155 reflects, in the regimen plan sheet, the content and the timing of treatment for dealing with the side effect in the next dosage period that are suggested or changed on the dosage regimen support screen. Specifically, in FIG. 5, the registration function 155 adds, to the regimen plan sheet, a plan for administering the antifebrile two days after and seven days after administration of the anticancer agent. The regimen plan sheet in which the suggestion is reflected by the registration function 155 is stored in the storage 120.

The user can also change the treatment plan on the regimen screen. For example, the user may move the timing of prescribing the antifebrile by a drag-and-drop operation on the regimen screen 80. The user may also select a medicine to be prescribed from a list box 72 on the regimen screen 80 to be changed or added.

Returning to FIG. 1, the following describes the input terminal 200.

The input terminal 200 includes a NW interface 210, a storage 220, an input interface 230, a display 240, and a processing circuit 250.

The processing circuit 250 is processing circuitry that implements a function corresponding to each computer program by reading out, from the storage 220, and executing the computer program. The processing circuit 250 according to the present embodiment includes a display control function 251, a reception function 252, and a transmission function 253. The display control function 251 is an example of a display control unit. The reception function 252 is an example of a reception unit. The transmission function 253 is an example of a transmission unit.

For example, respective processing functions of the display control function 251, the reception function 252, and the transmission function 253 as constituent elements of the processing circuit 250 are stored in the storage 220 as a computer-executable program. The processing circuit 250 is processing circuitry. For example, the processing circuit 250 implements a function corresponding to each computer program by reading out, from the storage 220, and executing the computer program. In other words, the processing circuit 250 that has read out each computer program is assumed to have each function illustrated in the processing circuit 250 in FIG. 1. Although in FIG. 1, a single piece of the processing circuitry including the display control function 251, the reception function 252, and the transmission function 253 implements the respective processing functions, a plurality of independent pieces of the processing circuitry may be combined to configure the processing circuit 250, so as to implement the functions when each piece of the processing circuitry executes the corresponding computer program. In FIG. 1, although the single storage 220 stores therein a computer program corresponding to each processing function, a plurality of storages may be disposed in a distributed manner, and the processing circuit 250 may be configured to read out a computer program from the corresponding storage.

The display control function 251 causes the display 240 to display an input screen with which the patient can input the report information.

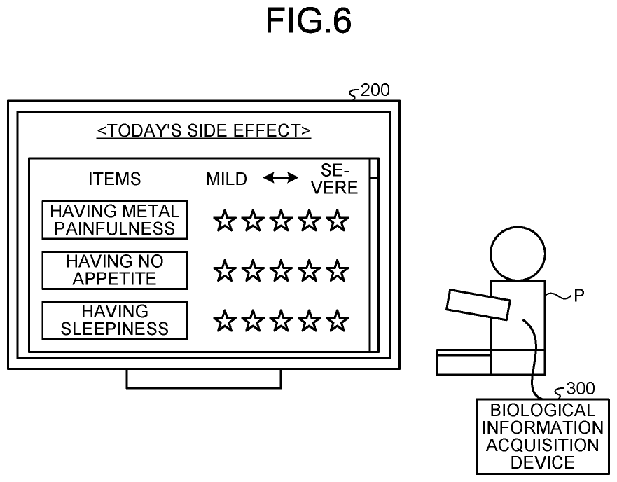

FIG. 6 is a diagram illustrating an example of the input screen for the report information according to the present embodiment. In the example illustrated in FIG. 6, the display control function 251 displays an input screen with which a degree of severity of the symptom for each day can be input at five levels regarding the side effect including “having mental painfulness”, “having no appetite”, and “having sleepiness”. A layout of the input screen and a method of evaluating the degree of severity of the symptom are not limited to the example illustrated in FIG. 6.

Returning to FIG. 1, the reception function 252 receives an input of the report information from a patient P. For example, the reception function 252 receives, as the report information, an input date and the degree of severity of the symptom for each type of the side effect that are input through the input screen illustrated in FIG. 6.

The reception function 252 sends out the received report information to the transmission function 253.

The transmission function 253 transmits the report information received by the reception function 252 to the dosage regimen supporting apparatus 100.

As illustrated in FIG. 6, the biological information acquisition device 300 is mounted on the patient P. As described above, the biological information acquisition device 300 is, for example, a thermometer, a sphygmomanometer, a pulse oximeter, or the like. The biological information acquisition device 300 transmits the biological information, which is obtained by measuring the patient P, to the dosage regimen supporting apparatus 100 via the network N, for example. A method of transmitting the biological information from the biological information acquisition device 300 to the dosage regimen supporting apparatus 100 is not limited.

Next, the following describes a processing procedure performed by the dosage regimen supporting apparatus 100 configured as described above.

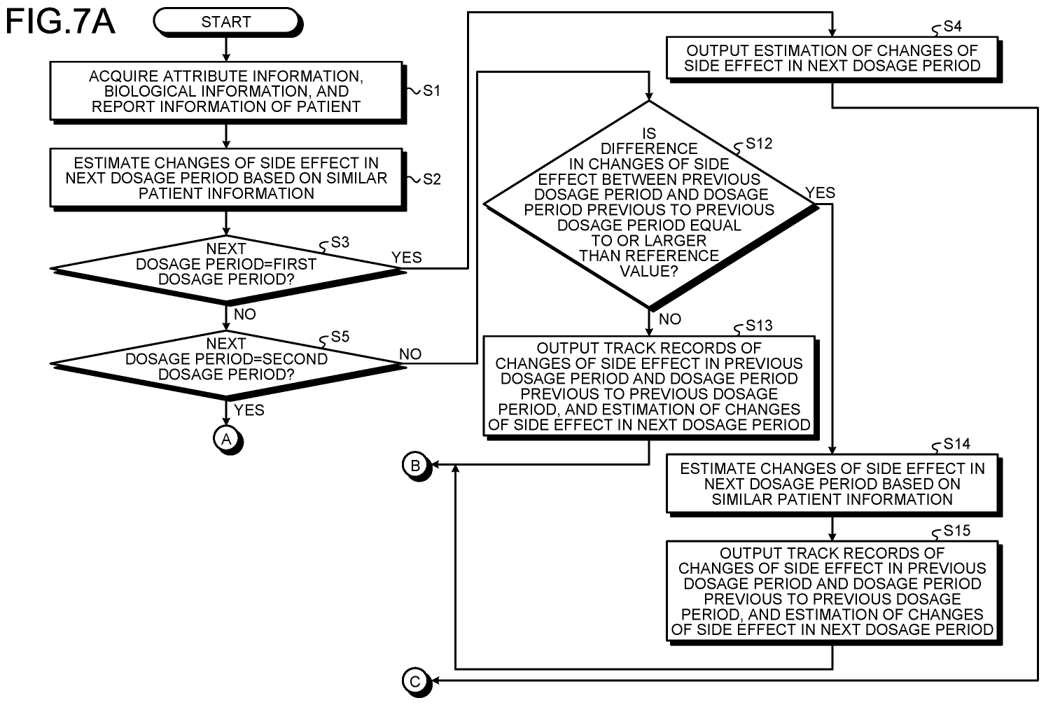

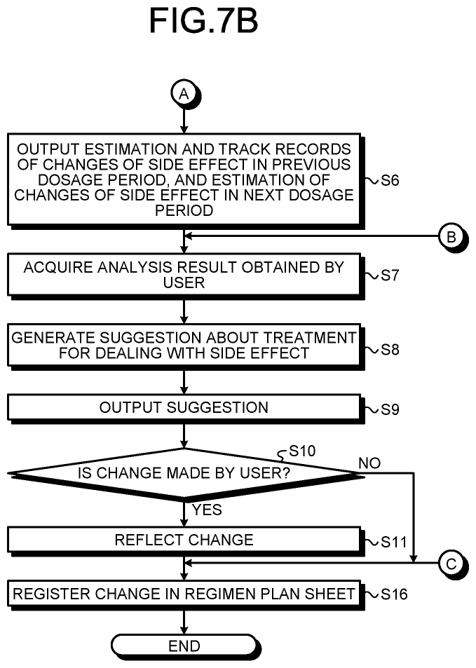

FIG. 7A, 7B is a flowchart illustrating an example of a procedure of dosage regimen support processing according to the present embodiment. First, the acquisition function 151 acquires the attribute information, the biological information, and the report information of the patient P (S1). The acquisition function 151 associates the acquired attribute information, biological information, and report information of the patient P with the identification information of the patient P to be stored in the storage 120.

Next, the estimation function 152 extracts the similar patient information from the storage 120 based on the acquired attribute information of the patient P. The estimation function 152 estimates changes of the side effect in the next dosage period based on the similar patient information (S2). The estimation function 152 outputs an estimation result to the generation function 153 and the output function 154.

Next, the output function 154 determines whether the next dosage period is the first dosage period (S3). For example, the output function 154 calculates the number of the next dosage period by counting the number of dosage periods that have been completed based on the regimen plan sheet or other records stored in the storage 120.

If it is determined that the next dosage period is the first dosage period (Yes at S3), the output function 154 outputs estimation of changes of the side effect in the next dosage period obtained by the estimation function 152 (S4). For example, the output function 154 causes the display 140 to display the dosage regimen support screen. In this case, track records are not present in a case in which the next dosage period is the first dosage period, so that only the estimated values generated from the similar patient information are displayed in the first area 61 of the dosage regimen support screen. In this case, the estimated values generated from the similar patient information are also displayed in the second area 62. Alternatively, in this case, the output function 154 may display only the second area 62 on the dosage regimen support screen, and does not necessarily display the first area 61. After the processing at S4, the process proceeds to the processing at S16.

If it is determined that the next dosage period is not the first dosage period (No at S3), the output function 154 determines whether the next dosage period is the second dosage period (S5).

If it is determined that the next dosage period is the second dosage period (Yes at S5), the output function 154 outputs estimation and track records of changes of the side effect in the previous dosage period, and estimation of changes of the side effect in the next dosage period (S6). For example, as described above with reference to FIG. 2, the output function 154 displays estimation and track records of changes of the side effect in the first dosage period in the first area 61 of the dosage regimen support screen, and displays estimation of changes of the side effect in the second dosage period in the second area 62.

Next, the acquisition function 151 acquires an analysis result obtained by the user (S7). For example, the acquisition function 151 acquires the analysis result input by the user in the first area 61 of the dosage regimen support screen.

The generation function 153 then generates a suggestion about treatment for dealing with the side effect in the next dosage period (S8). The suggestion about treatment for dealing with the side effect is a suggestion related to the content and the timing of treatment. For example, the generation function 153 generates a suggestion about a countermeasure based on the analysis result acquired at S7 and the biological information or the report information of the patient P in the first dosage period.

Next, the output function 154 outputs the suggestion generated by the generation function 153 (S9). Specifically, the output function 154 displays the suggestion generated by the generation function 153 in the second area 62 of the dosage regimen support screen.

Next, the acquisition function 151 determines whether the suggestion is changed by the user (S10).

If the user performs an operation of changing the suggestion (Yes at S10), the acquisition function 151 acquires content of the change. The generation function 153 reflects the change in the second area 62 of the dosage regimen support screen based on the content of the change acquired by the acquisition function 151 (S11). After the processing at S11, the process proceeds to the processing at S16.

If the suggestion is not changed by the user (No at S10), for example, in a case in which the user performs an operation of approving the suggestion generated by the generation function 153, the process proceeds to the processing at S16.

If the output function 154 determines that the next dosage period is not the second dosage period (No at S5), the estimation function 152 determines whether a difference between the changes of the side effect in the previous dosage period and the dosage period previous to the previous dosage period is equal to or larger than a reference value (S12).

If it is determined that the difference between changes of the side effect in the previous dosage period and the dosage period previous to the previous dosage period is smaller than the reference value (No at S12), the estimation function 152 estimates the symptom of the side effect of the patient P and the timing of onset of the symptom in the next dosage period based on the actual symptom of the side effect of the patient P and the timing of onset of the symptom in the previous dosage period.

In this case, the output function 154 outputs track records of changes of the side effect in the previous dosage period and the dosage period previous to the previous dosage period, and estimation of changes of the side effect in the next dosage period (S13). In this case, estimation of changes of the side effect in the next dosage period is estimation based on the actual symptom of the side effect of the patient P and the timing of onset of the symptom. For example, the output function 154 displays track records of changes of the side effect in the previous dosage period and the dosage period previous to the previous dosage period in the first area 61 of the dosage regimen support screen. The output function 154 also displays estimation of changes of the side effect in the next dosage period in the second area 62 of the dosage regimen support screen. After the processing at S13, the process proceeds to the processing at S7 described above.

If it is determined that the difference between the changes of the side effect in the previous dosage period and the dosage period previous to the previous dosage period is equal to or larger than the reference value (Yes at S12), the estimation function 152 estimates changes of the side effect in the next dosage period based on the similar patient information (S14).

The output function 154 then outputs track records of changes of the side effect in the previous dosage period and the dosage period previous to the previous dosage period, and estimation of changes of the side effect in the next dosage period (S15). For example, as described above with reference to FIG. 3, the output function 154 displays track records of changes of the side effect in the previous dosage period and the dosage period previous to the previous dosage period in the first area 61 of the dosage regimen support screen. The output function 154 displays estimation of changes of the side effect in the next dosage period in the second area 62 of the dosage regimen support screen. After the processing at S15, the process proceeds to the processing at S7 described above.

The registration function 155 then registers, in the regimen plan sheet, the content and the timing of treatment included in the suggestion generated by the generation function 153 (S16). In a case in which the suggestion is changed by the user, the registration function 155 registers the content or the timing of treatment changed by the user in the regimen plan sheet. At this point, the processing of this flowchart ends.

In this way, the dosage regimen supporting apparatus 100 according to the present embodiment acquires at least one of the biological information and the report information of the patient P, and the attribute information of the patient P, and outputs the suggestion related to the content and the timing of treatment for dealing with the side effect based on the biological information or the report information of the patient P. Thus, the present embodiment can support the doctor to make the treatment plan while considering the side effect that individually occurs in the patient P by supporting the doctor to incorporate the treatment for dealing with the side effect based on the suggestion into the treatment plan for administering the anticancer agent.

For example, in a case in which the doctor and the like make a treatment plan for the next dosage period, the regimen plan sheet is updated based on an examination result of the patient P or a self-reported symptom of the side effect in the previous dosage period. Appropriately setting the content and the timing of treatment for dealing with the side effect with reference to past records is not easy work, and requires technical training and experience. The dosage regimen supporting apparatus 100 according to the present embodiment outputs the suggestion related to the content and the timing of treatment for dealing with the side effect in accordance with the patient P, so that it is possible to reduce a load of examination work related to the content and the timing of treatment performed by the doctor.

The dosage regimen supporting apparatus 100 according to the present embodiment also outputs the estimation result of the symptom of the side effect and the timing of onset of the symptom based on the similar patient information, and the suggestion related to the content and the timing of treatment for dealing with the side effect based on the biological information or the report information of the patient P, and the estimation result. Typically, the characteristics such as the timing of onset of the side effect with the anticancer agent, severity of the symptom, and duration of the symptom are different among individuals, but these characteristics may be similar, with high possibility, between patients having similar pieces of attribute information. Thus, the present embodiment can support making the treatment plan while considering the side effect that individually occurs in the patient P by using the similar patient information.

In the present embodiment, the therapeutic agent is an agent that is periodically administered to the patient P for every dosage period having a predetermined length for multiple times. In a case in which such a therapeutic agent is administered, the dosage regimen supporting apparatus 100 according to the present embodiment estimates the symptom of the side effect of the patient P and the timing of onset of the symptom in the next dosage period based on the similar patient information, or the biological information or the report information in the previous dosage period of the patient P. Thus, the dosage regimen supporting apparatus 100 according to the present embodiment can support the doctor to make a plan while considering the side effect as occasion demands before each dosage period starts.

The dosage regimen supporting apparatus 100 according to the present embodiment generates the suggestion related to the content and the timing of treatment for dealing with the side effect based on the analysis result related to the side effect of the patient P in the previous dosage period, and the symptom of the side effect of the patient P and the timing of onset of the symptom in the previous dosage period. The dosage regimen supporting apparatus 100 according to the present embodiment displays, as the treatment plan for the next dosage period, a result obtained by superimposing the timing of treatment in the suggestion described above on time-series changes of the actual symptom of the side effect of the patient P in the previous dosage period or time-series changes of the symptom of the side effect of the patient P based on the estimation result. Due to this, the dosage regimen supporting apparatus 100 according to the present embodiment can enable the doctor to easily examine the content and the timing of treatment for dealing with the side effect of the patient P in the next dosage period together with the estimated value of the side effect of the patient P.

The analysis result according to the present embodiment is based on an analysis of the symptom of the side effect of the patient P and the timing of onset of the symptom in the previous dosage period by the doctor. Thus, the dosage regimen supporting apparatus 100 according to the present embodiment can generate a suggestion reflecting determination of the doctor.

In a case in which the symptom of the side effect of the patient P or the timing of onset of the symptom in the previous dosage period is different from the symptom of the side effect of the patient P or the timing of onset of the symptom in the dosage period previous to the previous dosage period by a predetermined reference value or more, the dosage regimen supporting apparatus 100 according to the present embodiment estimates the symptom of the side effect of the patient P and the timing of onset of the symptom in the next dosage period based on the information related to developments of the side effect of a similar patient. Thus, even in a case in which it is difficult to estimate the symptom of the side effect of the patient P in the next dosage period from the past information of the patient P, the dosage regimen supporting apparatus 100 according to the present embodiment can support the doctor to make the treatment plan for the next dosage period by estimating the side effect of the patient P in the next dosage period based on a similar patient.

In a case in which the symptom of the side effect of the patient P or the timing of onset of the symptom in the previous dosage period is not different from the symptom of the side effect of the patient P or the timing of onset of the symptom in the dosage period previous to the previous dosage period by the predetermined reference value or more, the dosage regimen supporting apparatus 100 according to the present embodiment estimates the symptom of the side effect of the patient and the timing of onset of the symptom in the next dosage period based on the symptom of the side effect of the patient P and the timing of onset of the symptom in the previous dosage period. As a characteristic of the side effect of the anticancer agent, typically, in a case in which the anticancer agent is administered to the same person over a plurality of dosage periods, the timing of onset of the side effect, severity of the symptom, and duration of the symptom are similar among the respective dosage periods. Due to this, the dosage regimen supporting apparatus 100 according to the present embodiment can estimate the side effect of the patient P in the next dosage period with high accuracy by effectively using the information related to the side effect of the patient P in the previous dosage period.

The dosage regimen supporting apparatus 100 according to the present embodiment registers the treatment plan based on the suggestion related to the content and the timing of treatment for dealing with the side effect in the regimen plan sheet including a time-series treatment plan related to administration of the anticancer agent. Due to this, the dosage regimen supporting apparatus 100 according to the present embodiment can facilitate manual work for changing the regimen plan sheet performed by the doctor and the like.

In the present embodiment, the doctor can change the content or the timing of treatment suggested by the dosage regimen supporting apparatus 100. The dosage regimen supporting apparatus 100 according to the present embodiment registers the content or the timing of treatment changed by the doctor in the regimen plan sheet. Thus, the doctor can appropriately change the content and the timing of treatment for dealing with the side effect suggested by the dosage regimen supporting apparatus 100 on his/her own judgment.

In the dosage regimen supporting system S according to the present embodiment, the input terminal 200 receives an input of the report information of the patient P as a dosage target of the therapeutic agent, and transmits the received report information to the dosage regimen supporting apparatus 100. Thus, the dosage regimen supporting system S according to the present embodiment can support generation of the treatment plan while considering a subjective side effect that is difficult to be quantitatively measured.

First Modification

In the embodiment described above, an example having a configuration in which the dosage regimen supporting apparatus 100 includes one PC or the like is described, but the dosage regimen supporting apparatus 100 may include a plurality of information processing devices. For example, the dosage regimen supporting apparatus 100 may include a server device, and an information processing device such as a PC or a tablet terminal connected to the server device via a network or the like. Part of the function of the dosage regimen supporting apparatus 100 may be disposed in a cloud environment.

The functions described as the functions of the input terminal 200 in the embodiment described above may be included in the dosage regimen supporting apparatus 100.

Second Modification

In the embodiment described above, the dosage regimen supporting apparatus 100 acquires both of the biological information and the report information, but may be configured to acquire only one of them.

Third Modification

In the embodiment described above, the report information is exemplified as the information indicating the symptom of the subjective side effect of the patient P, but other information may also be used as the information indicating the symptom of the subjective side effect of the patient P.

For example, the acquisition function 151 of the dosage regimen supporting apparatus 100 may acquire information indicating the content and the timing of the symptom complained by the patient P, or a condition of the patient P related to the side effect from a nursing record written by a nurse who attends the patient P, or an electronic medical chart input by the doctor.

For example, the processing circuit 150 of the dosage regimen supporting apparatus 100 may further include an estimation function for estimating a degree of severity of the symptom included in the report information input by the patient P based on information of the nursing record or the electronic medical chart. The estimation function is an example of an estimation unit.

For example, regarding a subjective symptom such as “having no appetite” among side effects, it is not easy to evaluate accuracy of severity of the symptom self-reported by the patient P. Thus, for example, the estimation function may acquire, from the nursing record, an actual dietary intake of the patient P on a day when the patient P inputs “having no appetite”, and estimate severity of the symptom of the side effect of “having no appetite” on this day. For example, in a case in which the actual dietary intake of the patient P is not reduced, the estimation function may estimate that the severity of the symptom of the side effect of “having no appetite” input by the patient P is low.

In a case in which the nursing record, the electronic medical chart, or the like includes a record about the symptom of the side effect answered by the patient P in response to an inquiry from the nurse or the doctor, the estimation function may estimate severity and the timing of the symptom of the subjective side effect of the patient P from the record. For example, in a case in which severity of the symptom of the side effect included in the report information input by the patient P is different from severity of the symptom of the side effect answered by the patient P in response to the inquiry from the nurse or the doctor described in the nursing record, the electronic medical chart, or the like, the estimation function may use the severity of the symptom of the side effect described in the nursing record, the electronic medical chart, or the like.

In a case in which the fact that treatment such as administration of a medicine or an intravenous drip is performed for dealing with the side effect is recorded in the electronic medical chart, the estimation function may estimate that the severity of the symptom of the side effect of the patient P is high. For example, in a case in which the fact that a nutrient is administered on an intravenous drip due to a decrease in dietary intake of the patient P is recorded in the electronic medical chart, the estimation function may estimate that the severity of the symptom of “having no appetite”, which is the side effect associated with the treatment, is high.

For example, the output function 154 may display, as a track record value of the side effect, the severity of the symptom of the side effect of the patient P based on the estimation result obtained by the estimation function. In the present modification, the estimation function may estimate the severity of the symptom of the side effect based on both of the report information input by the patient P and the information included in the nursing record or the electronic medical chart, or may estimate the severity of the symptom of the side effect based on only the information included in the nursing record or the electronic medical chart. In a case of estimating the severity of the symptom of the side effect based on only the information included in the nursing record or the electronic medical chart, the report information is not necessarily input by the patient P.

In a case in which the information included in the nursing record or the electronic medical chart is unstructured data such as a natural sentence, the information may be converted into structured data by the estimation function, for example. For example, in a case in which the degree of severity of the symptom is written as a natural sentence, the estimation function may convert the natural sentence into a score representing the degree of severity of the symptom. To convert unstructured data into structured data, a pre-learned model generated by deep learning and the like can be used, for example, but another method may also be used.

Fourth Modification

In the embodiment described above, it is assumed that only the estimated values generated from the similar patient information may be displayed on the dosage regimen support screen at the time of planning the first dosage period. However, in the first dosage period, the doctor and the like may give the patient P treatment based on the guideline without using the dosage regimen support screen. In this case, the doctor and the like use the dosage regimen support screen at the time of making a plan of the second dosage period.

Fifth Modification

In the embodiment described above, the generation function 153 generates the suggestion about the treatment for dealing with the side effect in the next dosage period, but the content of the suggestion is not limited thereto.

For example, the acquisition function 151 of the dosage regimen supporting apparatus 100 may further include a determination function for determining whether to start the next dosage period, or content of treatment recommended to be performed before the next dosage period based on the biological information or the report information of the patient P. The determination function is an example of a determination unit.

For example, based on an amount of ascites of the patient P, presence/absence of consciousness disorder, a degree of deterioration of a cardiac function, and the like, the determination function may determine that the next dosage period should be cancelled or postponed. The amount of ascites may be measured by abdominal echo and the like, for example, or may be acquired from findings input to the electronic medical chart by the doctor. The amount of ascites is assumed to be included in the biological information according to the present modification.

The determination function may determine content of treatment that should be performed before the next dosage period is started based on the biological information or the report information of the patient P. For example, in a case in which the amount of ascites of the patient P is equal to or larger than a reference value, the determination function may determine that the content of treatment that should be performed is to extract ascites before the next dosage period is started.

In the present modification, the output function 154 outputs a determination result obtained by the determination function. For example, the output function 154 may cause the display 140 to display whether to start the next dosage period, or the content of treatment that is recommended to be performed before the next dosage period.

Processing of the determination function may be implemented as a function of the generation function 153. For example, the generation function 153 may generate a suggestion to cancel or postpone the next dosage period based on the biological information or the report information of the patient P. The generation function 153 may also generate a suggestion about treatment that should be performed before the next dosage period is started based on the biological information or the report information of the patient P.

Sixth Modification

In the embodiment described above, the analysis result is a result of analysis performed by the doctor, but the analysis result is not limited thereto.

For example, the processing circuit 150 of the dosage regimen supporting apparatus 100 may further include an analysis function for performing analysis of the symptom of the side effect of the patient P and the timing of onset of the symptom in the previous dosage period. The analysis function is an example of an analysis unit.

For example, the analysis function may generate, as an analysis result, a difference between estimation and track records of severity or the timing of the symptom of the side effect in the previous dosage period. The analysis function may also generate, as the analysis result, a difference between the severity or the timing of the symptom of the side effect in the dosage period previous to the previous dosage period and severity or the timing of the symptom of the side effect in the previous dosage period.

In the present modification, the generation function 153 generates the suggestion related to the content and the timing of treatment for dealing with the side effect based on the analysis result obtained by the analysis function.

Seventh Modification

Means for inputting the report information is not limited to a screen input. For example, in a case in which the input terminal 200 is a smartphone, the patient P may image a face of himself/herself to input a face photograph. In this case, for example, the processing circuit 250 of the input terminal 200 may determine whether the patient P has jaundice based on the face photograph to be transmitted to the dosage regimen supporting apparatus 100.

According to at least one of the embodiments described above, it is possible to support making a treatment plan while considering a side effect that individually occurs in a patient.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A dosage regimen supporting apparatus comprising:
processing circuitry configured to
acquire attribute information of a patient as a dosage target of a therapeutic agent, and at least one of quantitative biological information indicating a symptom of a side effect caused by the therapeutic agent for the patient and a timing of onset of the symptom, and subjective report information of the patient related to the symptom of the side effect and the timing of onset of the symptom,
estimate the symptom of the side effect of the patient and the timing of onset of the symptom in a next dosage period of the therapeutic agent based on the biological information or the report information, and the attribute information of the patient, and
output a suggestion related to content and a timing of treatment for dealing with the side effect in the next dosage period based on the biological information or the report information, and an estimation result.

2. The dosage regimen supporting apparatus according to claim 1, wherein
the processing circuitry is configured to
estimate, based on information related to the symptom of the side effect and the timing of onset of the symptom of a similar patient having attribute information similar to the attribute information of the patient, and
cause a display unit to display the estimation result in a comparable manner with the biological information or the report information of the patient on a time-series basis.

3. The dosage regimen supporting apparatus according to claim 1, wherein
the therapeutic agent is an agent that is periodically administered to the patient for every dosage period having a predetermined length for multiple times, and
the processing circuitry is configured to estimate the symptom of the side effect of the patient and the timing of onset of the symptom in the next dosage period based on the biological information or the report information of the patient in a previous dosage period.

4. The dosage regimen supporting apparatus according to claim 1, wherein
the processing circuitry is configured to
generate the suggestion based on an analysis result related to the side effect of the patient in a previous dosage period, and the symptom of the side effect of the patient and the timing of onset of the symptom in the previous dosage period, and
cause a display unit to display the timing of the treatment in the suggestion so as to be superimposed on time-series changes of the symptom of the side effect of the patient in the previous dosage period, or time-series changes of the symptom of the side effect of the patient based on the estimation result.

5. The dosage regimen supporting apparatus according to claim 4, wherein the analysis result is based on analysis by a doctor related to the symptom of the side effect of the patient and the timing of onset of the symptom in the previous dosage period.

6. The dosage regimen supporting apparatus according to claim 4, wherein
the processing circuitry is configured to
analyze the symptom of the side effect of the patient and the timing of onset of the symptom in the previous dosage period, and
generate the suggestion based on an analysis result.

7. The dosage regimen supporting apparatus according to claim 2, wherein
the processing circuitry is configured to
estimate the symptom of the side effect of the patient and the timing of onset of the symptom in the next dosage period based on information related to developments of the side effect of the similar patient in a case in which the symptom of the side effect of the patient or the timing of onset of the symptom in a previous dosage period is different from the symptom of the side effect of the patient or the timing of onset of the symptom in a dosage period previous to the previous dosage period by a predetermined reference value or more, and
estimate the symptom of the side effect of the patient and the timing of onset of the symptom in the next dosage period based on the symptom of the side effect of the patient and the timing of onset of the symptom in the previous dosage period in a case in which the symptom of the side effect of the patient and the timing of onset of the symptom in the previous dosage period is not different from the symptom of the side effect of the patient or the timing of onset of the symptom in the dosage period previous to the previous dosage period by the predetermined reference value or more.

8. The dosage regimen supporting apparatus according to claim 1, wherein
the therapeutic agent is an anticancer agent, and
the processing circuitry is configured to register a treatment plan based on the suggestion in a regimen plan sheet including a time-series treatment plan related to administration of the anticancer agent.

9. The dosage regimen supporting apparatus according to claim 8, wherein
the processing circuitry is configured to
acquire a change of the content or the timing of the treatment in the suggestion, the change being made by a user, and
register the content or the timing of the treatment changed by the user in the regimen plan sheet.

10. The dosage regimen supporting apparatus according to claim 1, wherein
the processing circuitry is configured to
determine whether to start the next dosage period, or content of treatment that is recommended to be performed before the next dosage period based on the biological information or the report information, and
output a determination result.

11. The dosage regimen supporting apparatus according to claim 2, wherein at least one of an age, distinction of sex, a physical constitution, a body mass index (BMI), a type of cancer, a stage of cancer, vital data at the time when treatment is started with the therapeutic agent, and a chronic disease is same between the similar patient and the patient.

12. A dosage regimen supporting system comprising an input terminal and a dosage regimen supporting apparatus, wherein
the input terminal comprises first processing circuitry configured to
receive an input of subjective report information of a patient as a dosage target of a therapeutic agent, related to a symptom of a side effect caused by the therapeutic agent for the patient and the timing of onset of the symptom, and
transmit the received report information to the dosage regimen supporting apparatus, and
the dosage regimen supporting apparatus comprises second processing circuitry configured to
acquire attribute information of the patient, and at least one of the report information and quantitative biological information indicating the symptom of the side effect caused by the therapeutic agent for the patient and the timing of onset of the symptom,
estimate the symptom of the side effect of the patient and the timing of onset of the symptom in a next dosage period of the therapeutic agent based on the biological information or the report information, and the attribute information of the patient, and
output a suggestion related to content and a timing of treatment for dealing with the side effect based on the biological information or the report information, and an estimation result.

13. The dosage regimen supporting system according to claim 12, wherein
the second processing circuitry is configured to
estimate the symptom of the side effect of the patient and the timing of onset of the symptom based on information about the symptom of the side effect and the timing of onset of the symptom of a similar patient having attribute information similar to the attribute information of the patient, and
cause a display unit to display the estimation result in a comparable manner with the biological information or the report information of the patient on a time-series basis.

14. The dosage regimen supporting system according to claim 12, wherein
the therapeutic agent is an agent that is periodically administered to the patient for every dosage period having a predetermined length for multiple times, and
the second processing circuitry is configured to estimate the symptom of the side effect of the patient and the timing of onset of the symptom in the next dosage period based on the biological information or the report information of the patient in a previous dosage period.

15. The dosage regimen supporting system according to claim 12, wherein
the second processing circuitry is configured to
generate the suggestion based on an analysis result related to the side effect of the patient in a previous dosage period, and the symptom of the side effect of the patient and the timing of onset of the symptom in the previous dosage period, and
cause a display unit to display the timing of the treatment in the suggestion so as to be superimposed on time-series changes of the symptom of the side effect of the patient in the previous dosage period, or time-series changes of the symptom of the side effect of the patient based on the estimation result.

16. The dosage regimen supporting system according to claim 15, wherein the analysis result is based on analysis that is performed by a doctor related to the symptom of the side effect of the patient and the timing of onset of the symptom in the previous dosage period.

17. The dosage regimen supporting system according to claim 15, wherein
the second processing circuitry is configured to
analyze the symptom of the side effect of the patient and the timing of onset of the symptom in the previous dosage period, and
generate the suggestion based on an analysis result.

18. The dosage regimen supporting system according to claim 13, wherein
the second processing circuitry is configured to
estimate the symptom of the side effect of the patient and the timing of onset of the symptom in the next dosage period based on information related to developments of the side effect of the similar patient in a case in which the symptom of the side effect of the patient or the timing of onset of the symptom in a previous dosage period is different from the symptom of the side effect of the patient or the timing of onset of the symptom in a dosage period previous to the previous dosage period by a predetermined reference value or more, and
estimate the symptom of the side effect of the patient and the timing of onset of the symptom in the next dosage period based on the symptom of the side effect of the patient and the timing of onset of the symptom in the previous dosage period in a case in which the symptom of the side effect of the patient and the timing of onset of the symptom in the previous dosage period is not different from the symptom of the side effect of the patient or the timing of onset of the symptom in the dosage period previous to the previous dosage period by the predetermined reference value or more.

19. The dosage regimen supporting system according to claim 12, wherein
the therapeutic agent is an anticancer agent, and
the second processing circuitry is configured to register a treatment plan based on the suggestion in a regimen plan sheet including a time-series treatment plan related to administration of the anticancer agent.

20. The dosage regimen supporting system according to claim 19, wherein
the second processing circuitry is configured to
acquire a change of the content or the timing of the treatment in the suggestion, the change being made by a user, and
register the content or the timing of the treatment changed by the user in the regimen plan sheet.
